(19) United States
(12) Patent Application Publication
(10) Pub. No.: US 2017/0244110 A1
(43) Pub. Date: Aug. 24, 2017

(54) INTEGRATED METHODS AND SYSTEMS FOR ELECTRICAL MONITORING OF CANCER CELLS STIMULATED BY ELECTROMAGNETIC WAVES

(71) Applicants: Mohammad Abdolahad, Tehran (IR); Saeed Rafizadeh Tafti, Yazd (IR)

(72) Inventors: Mohammad Abdolahad, Tehran (IR); Saeed Rafizadeh Tafti, Yazd (IR)

(21) Appl. No.: 15/588,599

(22) Filed: May 6, 2017

Related U.S. Application Data
(60) Provisional application No. 62/333,295, filed on May 9, 2016.

(51) Int. Cl. H01M 4/90; B82Y 30/00; H01M 8/16; B82Y 10/00; H01M 8/04186; C12Q 1/00

(52) U.S. Cl. CPC H01M 4/9083; H01M 8/04194; C12Q 1/001; H01M 8/16; B82Y 10/00; B82Y 30/00; H01M 4/8647

(57) ABSTRACT

A method for stimulating and analyzing of cancer cells, including: preparing an integrated stimulating-analyzing set-up including an array of carbon nanotubes (CNTs), measuring a first electrical response from the attached cancer cells, applying an electromagnetic field on the attached cancer cells to stimulate cancer cells, measuring a second electrical response from the stimulated cancer cells, and detecting the vitality of the stimulated cancer cells by comparing the first and the second measured electrical responses.

### CROSS REFERENCE TO RELATED APPLICATION

The present application claims priority from pending U.S. Provisional Patent Application Ser. No. 62/333,295, filed May 9, 2016, entitled “Vertically aligned carbon nanotube based electrical bio-chip to transfer and detect electromagnetic stimulation on the cells”, which is incorporated by reference herein in its entirety.

### TECHNICAL FIELD

The present application generally relates to concurrent electromagnetic stimulation and monitoring the electrical behavior of cancer cells, using a biosensor based on the carbon nanotubes (CNTs) properties.

### BACKGROUND

Membrane voltage and charge states play a crucial role in the regulation of living cells' functionality. For example, a cell exposed to a strong external electric field, which experiences a build-up of opposing ion charges across its membrane, may fail to maintain cellular equilibrium and enter to apoptotic phase. The impact of transmembrane charge accumulation on cellular vitality strongly depends on the intensity and duration of the stimulations. Controlling the intensity of such phenomena to maintain the cell in living state or transforming it to apoptosis, is a challenge which requires many biological assays such as 3-(4,5-dimethylthiazol-2-yl)-2,5-diphenyltetrazolium bromide (MTT), flowcytometry, etc.

Producing localized charge accumulation on cell membrane would cast new lights in investigating the charge-cell interactions applicable in diagnostic and therapeutic purposes. Vertically aligned carbon nanotubes (VACNTs) as nano-scale field enhancers are good candidates in producing localized charge accumulation under the exposure of electromagnetic (EM) wave. Cell's transmembrane electrical environment would be highly affected in direct interaction with wave-stimulated CNTs. In addition, nanotubes could penetrate into the cell inner parts without damaging its membrane as previously reported by our group. Moreover, CNT based bioelectronics has been considerably developed in recent years since it exhibited anomalous physical and electrical properties.

There are many kinds of CNT-based biochips. Some of them are based on interacting with chemical agents in solutions as CNT's provide larger interacting area than smooth surfaces, for instance, in many biomedical ligand-based applications. In some applications, CNTs have been used widely in DNA-based biochips due to nano-geometry and deflection properties of CNTs. In another group of biosensors, vertically aligned CNT's have been applied as both entrapping sites for cancer cells and signal extracting probes from the grasped cells. Based on the extracted signals, several diagnostic approaches were proposed.

Therefore, there is a need for a biosensor and the related methods and systems for transferring and enhancing external EM stimulation on the cells and detecting the EM irradiation on the cancer cells. Such biosensors, methods and systems would be useful for future diagnostic and therapeutic applications, such as wave-guided breakage and destruction of drug-resistance cancer cells.

### SUMMARY

In one general aspect of the present disclosure, an exemplary method for stimulating and analyzing of cancer cells is disclosed. The method may include the steps of: preparing an integrated stimulating-analyzing set-up that may include an array of carbon nanotubes (CNTs), where a plurality of cancer cells attached onto the array of CNTs, measuring a first electrical response from the attached cancer cells, applying an electromagnetic field on the attached cancer cells to stimulate cancer cells, measuring a second electrical response from the stimulated cancer cells and detecting the vitality of the stimulated cancer cells by comparing the first and the second measured electrical responses.,

In an exemplary implementations, the vitality of the stimulated cancer cells may be decreased if the second electrical response has a reversed trend versus the trend of the first electrical response in a same frequencies and intensities range of the applied electromagnetic field. In some examples, the vitality of the stimulated cancer cells may be decreased if the second electrical response has greater amounts versus the amounts of the first electrical response in a same range of applied frequencies and intensities. In some examples, the greater amounts of the second electrical response may be at least about 10 percent greater than the amounts of the first electrical response.

In one exemplary implementation, the carbon nanotubes (CNTs) may include vertically aligned multiwall carbon nanotubes (VAMWCNTs).

In one exemplary implementation, the integrated stimulating-analyzing set-up may include: a biosensor that may include an array of CNTs grown on a chip, an electrical analyzing device and an electromagnetic wave exposure device. The electrical analyzing device may include a data acquisition instrument that may be configured to send an electrical signal to the biosensor and receive an electrical response from the biosensor and a data processor that may be configured to process the received electrical signals. The biosensor, the data acquisition instrument and the data processor may be electrically connected. The electromagnetic wave exposure device may include a wave irradiator module and a frequency generator, where the frequency generator is connected to the wave irradiator module.

In one exemplary implementation, the integrated stimulating-analyzing set-up may be prepared via a method that may include: holding a biosensor that may include the array of CNTs in a sealed package, connecting the biosensor to an electrical analyzing device, inserting a solution of cancer cells into the sealed package and on the array of CNTs, placing the sealed package in an electromagnetic wave exposure device and placing the electromagnetic wave exposure device including the sealed package in an incubator.

In one exemplary implementation, the solution of cancer cells may include a plurality of cancer cells suspended in a cell-culture media. The cancer cells may include lung cancer cells or lung cancer cell lines.

In one exemplary implementation, the first and the second electrical responses may be measured within a determined frequency range between about 0.1 and about 500 kHz. The first and the second measured electrical responses may include a first set and a second set of electrical impedance values measured in the determined frequency range.

In one exemplary implementation, the electromagnetic field may be applied with an intensity in a range of about 1 dbm to about 20 dbm and with a frequency of about 940 MHz.

In another exemplary embodiment consistent with the present disclosure, a system for stimulating and analyzing of cancer cells is disclosed. The system may include: a biosensor that may include an array of carbon nanotubes (CNTs), an electromagnetic wave exposure mechanism and an electrical mechanism.

In one implementation, the electromagnetic wave exposure mechanism may include a wave irradiator module and a frequency generator. Where, the frequency generator may be connected to the wave irradiator module and the biosensor that may be placed in a sealed package and the sealed package may be placed within the wave irradiator module.

In one implementation, the electrical mechanism may include a data acquisition instrument that may be configured to send an electrical signal to the biosensor and receive an electrical response from the biosensor and a data processor that may be configured to process the received electrical signals. The biosensor, the data acquisition instrument and the data processor are electrically connected.

In one implementation, the biosensor may concurrently transfer electromagnetic stimulation to the cancer cells and acquire electrical signal from cancer cells. The carbon nanotubes (CNTs) may include vertically aligned multiwall carbon nanotubes (VAMWCNTs) and the sealed package may include a plaxy-glass set.

In one implementation, the biosensor may include: a substrate layer that may include a layer of silicon (Si), an insulator layer that may include a layer of silicon dioxide (SiO₂) formed on the substrate layer, a catalyst layer that may include a patterned layer of Nickel (Ni) and an array of carbon nanotubes (CNTs) on the patterned layer that may include an array of vertically aligned multiwall carbon nanotubes (VAMWCNTs) grown on the patterned catalyst layer.

### BRIEF DESCRIPTION OF THE DRAWINGS

FIG. lA illustrates an example of a method for stimulating and analyzing of cancer cells, consistent with one or more exemplary embodiments of the present disclosure.

FIG. 1B illustrates an example of a process for the preparing the integrated stimulating-analyzing set-up, consistent with one or more exemplary embodiments of the present disclosure.

FIG. 2A illustrates a schematic of an example of a single integrated system for both electromagnetic stimulating and electrical analyzing of cancer cells, consistent with one or more exemplary embodiments of the present disclosure.

FIG. 2B illustrates an example of a biosensor, in which a plurality of cancer cells attached onto an array of carbon nanotubes (CNTs), consistent with one or more exemplary embodiments of the present disclosure.

FIGS. 3A-3C illustrate exemplary steps of a method for designing and fabricating a biosensor including an array of CNTs onto a chip, consistent with one or more exemplary embodiments of the present disclosure.

FIG. 4A illustrates a field emission scanning electron microscope (FESEM) micrograph of an example section of the surface of a biosensor having an array of grown CNTs on the patterned Ni layer, consistent with one or more exemplary embodiments of the present disclosure.

FIG. 4B illustrates a field emission scanning electron microscope (FESEM) micrograph of an example magnified section of FIG. 4A, which illustrates an example array of the grown VAMWCNTs on the patterned Ni layer, consistent with one or more exemplary embodiments of the present disclosure.

FIG. 4C illustrates a field emission scanning electron microscope (FESEM) micrograph of an example grown array of CNTs, consistent with one or more exemplary embodiments of the present disclosure.

In FIG. 4D illustrates a transmission electron microscope (TEM) image of an example of one CNT, consistent with one or more exemplary embodiments of the present disclosure.

FIG. 5 illustrates a field emission scanning electron microscope (FESEM) of exemplary CNTs that are grown on the biosensor patterned surface and are attached and penetrated into the QUDB cells, consistent with one or more exemplary embodiments of the present disclosure.

FIG. 6A illustrates a norm of impedance variations diagram of the cells seeded on CNTs grown sensors about 4 hours after culturing with respect to 2 hours after culturing for the control non irradiated sample, consistent with one or more exemplary embodiments of the present disclosure.

FIG. 6B illustrates a norm of impedance variations diagram of the cells seeded on CNTs grown sensors about 4 hours after culturing with respect to 2 hours after culturing for a samples ablated to EM wave with an intensity of about 1 dbm, consistent with one or more exemplary embodiments of the present disclosure.

FIG. 6C illustrates a norm of impedance variations diagram of the cells seeded on CNTs grown sensors about 4 hours after culturing with respect to 2 hours after culturing for a samples ablated to EM wave with an intensity of about 7 dbm, consistent with one or more exemplary embodiments of the present disclosure.

FIG. 6D illustrates a norm of impedance variations diagram of the cells seeded on CNTs grown sensors about 4 hours after culturing with respect to 2 hours after culturing for a samples ablated to EM wave with an intensity of about 10 dbm, consistent with one or more exemplary embodiments of the present disclosure.

FIG. 7A illustrates an impedance phase variations diagram from 2 hours to 4 hours after culturing process for QUDB cells exposed to 10 dbm EM wave, consistent with one or more exemplary embodiments of the present disclosure.

FIG. 7B illustrates an impedance phase variations diagram from 2 hours to 4 hours after culturing process for non-irradiated QUDB control cells, consistent with one or more exemplary embodiments of the present disclosure.

FIG. 8A illustrates a field emission scanning electron microscope (FESEM) of exemplary non-irradiated QUDB cells attached to the CNTs array, consistent with one or more exemplary embodiments of the present disclosure.

FIG. 8B illustrates a field emission scanning electron microscope (FESEM) of exemplary QUDB cells attached to the CNTs array and ablated to a 1 dbm EM wave, consistent with one or more exemplary embodiments of the present disclosure.

FIG. 8C illustrates a field emission scanning electron microscope (FESEM) of exemplary QUDB cells attached to the CNTs array and ablated to a 7 dbm EM wave, consistent with one or more exemplary embodiments of the present disclosure.

FIG. 8D illustrates a field emission scanning electron microscope (FESEM) of exemplary QUDB cells attached to the CNTs array and ablated to a 10 dbm EM wave, consistent with one or more exemplary embodiments of the present disclosure.

FIG. 9 illustrates MTT results from the vitality of the cells seeded on CNT and Ni helical electrodes 2 hours after the wave ablation for about 5 min (4 hours after the cell culture) with respect to non-irradiated sensors and for a main control sample (seeded on coverslip).

### DETAILED DESCRIPTION

The following detailed description is presented to enable a person skilled in the art to make and use the methods and devices disclosed in exemplary embodiment of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Disclosed herein is an exemplary method to electrically monitor the effect of EM wave stimulation on CNT-penetrated cancer cells. Here, the CNT arrays may be grown in the architecture of helical electrodes as an impedimetric biosensor to detect the vitality of the cells exposed by EM wave. A homogenous electrode surface may be achieved for the cells to spread on the CNTs. Then, the whole package may be stimulated by EM wave in a frequency of about 940 MHz (standard frequency introduced by International Commission on Non-Ionizing Radiation Protection (ICNIRP) in 1998).

Overall, the CNT arrays may be applied as both charge based stimulators of cellular membrane by external EM wave and impedance based sensors to monitor any variations in cells' vitality. The excess charges induced on cancer cells, for example, lung cancer cells attached to stimulated CNTs, would affect cellular functionality. The intensity of the EM wave may have a key role in the amount of charge perturbations on CNT-cell composites which determine the destructive strength of ionic exchanges in cells' vitality. In the present disclosure, this may be investigated by impedance signals and may be approved by some analysis, for example, florescent and field emission scanning electron microscopies (FE-SEM) as well as biological assays such as MTT.

Furthermore, a system and a biosensor is disclosed to electrically monitor the effect of EM wave stimulation on CNT-penetrated cancer cells. This biosensor, which is a lab-on-chip may provide an effective tool for controlling cell membrane permeability, transmembrane potential and levels of apoptosis caused by EM wave in future therapeutic applications.

The disclosed vitality assessment by impedance spectroscopy can be used as a diagnostic bio-chip for prognosis and diagnosis of cancer. Also, the methods and systems of the present disclosure may be used for cancer therapeutic treatments utilizing a biosensor based on CNTs to penetrate into living cancer cells and to transfer and enhance EM waves resulting in cancer cells destruction that may be investigated by the changes in cell electrical signals.

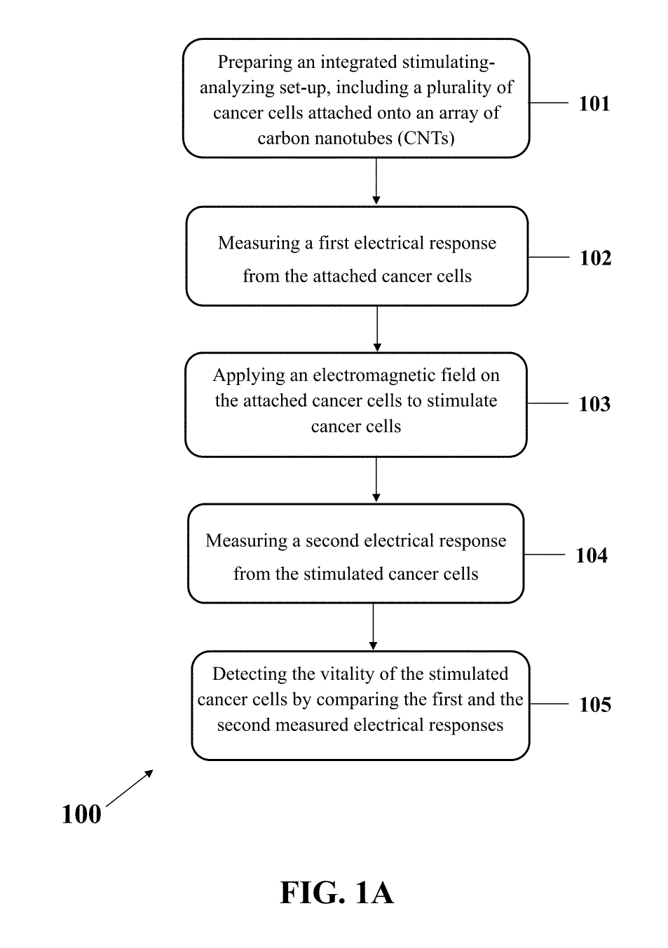

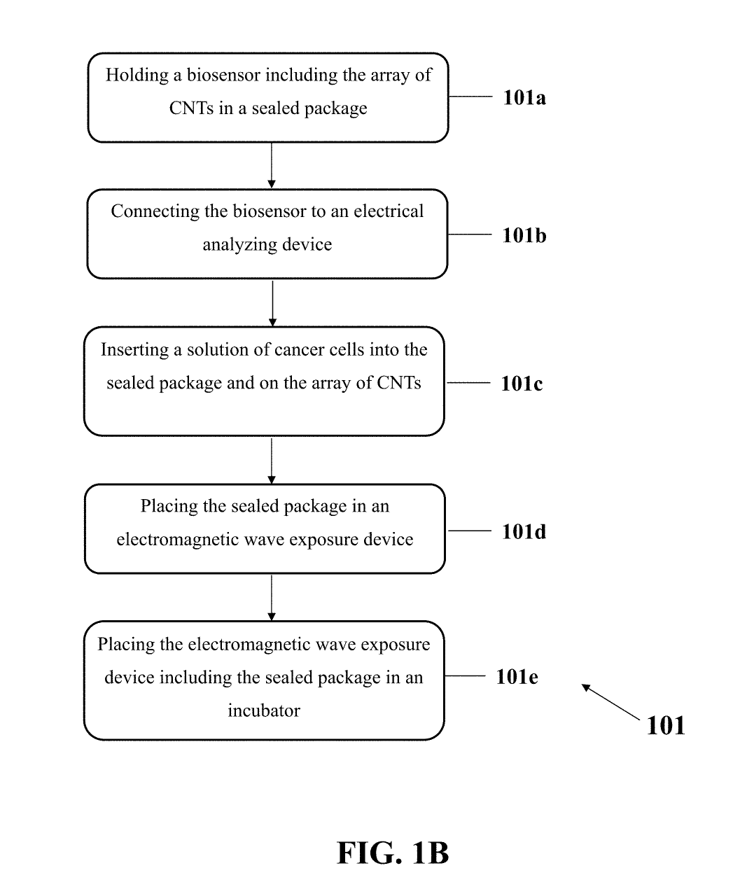

FIG. 1A shows an exemplary method 100 for stimulating and analyzing of cancer cells, consistent with one or more exemplary embodiments of the present disclosure. Method 100 may include the steps of preparing an integrated stimulating-analyzing set-up, including an array of carbon nanotubes (CNTs), where a plurality of cancer cells may be attached to the array of CNTs (step 101), measuring a first electrical response from the attached cancer cells (step 102), applying an electromagnetic field on the attached cancer cells to stimulate cancer cells (step 103), measuring a second electrical response from the stimulated cancer cells (step 104), and detecting the vitality of the stimulated cancer cells by comparing the first and the second measured electrical responses (step 105).

In step 101, an integrated stimulating-analyzing set-up may be prepared. The integrated stimulating-analyzing set-up may include an array of carbon nanotubes (CNTs). Where, a plurality of cancer cells that would be stimulated and analyzed may be attached onto the array of carbon nanotubes (CNTs). The carbon nanotubes (CNTs) may include vertically aligned multiwall carbon nanotubes (VAMWCNTs).

In some implementations, the integrated stimulating-analyzing set-up may be prepared through a method 101 that is shown in FIG. 1B. Referring to FIG. 1B, an exemplary integrated stimulating-analyzing set-up may be prepared by an example method 101 that may be included: holding a biosensor in a sealed package (step 101a), connecting the biosensor to an electrical analyzing device (step 101b), inserting a solution of cancer cells into the sealed package and on the array of CNTs (step 101c), placing the sealed package in an electromagnetic wave exposure device (step 101d), and placing the electromagnetic wave exposure device including the sealed package in an incubator (step 101e). The biosensor may include an array of CNTs, for example, an array of VAMWCNTs.

In some implementations, the integrated stimulating-analyzing set-up may include: a biosensor, including the array of CNTs grown on a chip, an electrical analyzing device, and an electromagnetic wave exposure device. The biosensor and the electrical analyzing device may be electrically connected.

In some implementations, the electrical analyzing device may include a data acquisition instrument and a data processor. The data acquisition instrument may be configured to send an electrical signal to the biosensor and receive an electrical response from the biosensor and the data processor may be configured to process the received electrical signals. So the biosensor, the data acquisition instrument and the data processor may be electrically connected.

In some implementations, the electromagnetic wave exposure device may include a wave irradiator module and a frequency generator. The frequency generator may be connected to the wave irradiator module.

In step 101c, a solution of cancer cells may be inserted into the sealed package and on the array of CNTs. The cancer cells may include lung cancer cells that may be resected from a patient or lung cancer cell lines, for example, QUDB cell lines that may be supplied from a cell bank. In this step, a solution of cancer cells that may include a plurality of cancer cells suspended in a cell-culture media may be dropped into the sealed package and on the array of CNTs.

In step 102, a first electrical response from the attached cancer cells onto the array of CNTs prepared in step 101, may be measured. The first electrical response may be an electrical signal received from the attached cells onto the CNTs array of the biosensor, for example electrical impedance or phase.

In step 103, an electromagnetic field may be applied on the attached cancer cells to stimulate cancer cells. The electromagnetic field may be applied to the attached cancer cells using the electromagnetic wave exposure device. The electromagnetic field may be applied with an intensity in a range of about 1 dbm to about 20 dbm.

In step 104, a second electrical response from the stimulated cancer cells after EM field applying in step 103 may be measured. The second electrical response may be an electrical signal received from the stimulated cells on the CNTs array of the biosensor, for example electrical impedance or phase.

In some implementation, the first and the second electrical responses may be measured within a determined frequency range between about 0.1 and about 500 kHz to obtain a set of electrical responses forming a trend on electrical responses within the determined frequency range. So the first and the second measured electrical responses may include a first set and a second set of electrical impedance values measured in the determined frequency range before and after the EM field applying on the cancer cells attached onto the CNTs array of the biosensor.

In step 105, the vitality of the stimulated cancer cells may be detected by comparing the first and the second measured electrical responses in steps 102 and 104. The vitality of the stimulated cancer cells may be a criterion to determine whether the EM waves exposure to the cancer cells affect them or not. If the second electrical response has a reversed trend in comparison with the trend of the first electrical response in the same frequencies and intensities of the applied electromagnetic field, the vitality of the stimulated cancer cells may be decreased or the cancer cells may be destructed because of the destructive EM waves effects on cancer cells. For example, if the second electrical response has a rising trend while the first electrical response has a falling trend so a cancer cell destruction may be occurred.

In some implementations, the amount or value of electrical responses may be a criterion to determine whether the EM waves exposure to the cancer cells affect the cancer cells, for example, cancer cells vitality or not. For example, if the second electrical response has greater amounts versus the amounts of the first electrical response in a same range of applied frequencies and intensities, the vitality of the stimulated cancer cells may be decreased. In some specific examples, wherein the greater amounts of the second electrical response may be at least 10 percent (10%) greater than the amounts of the first electrical response if the cancer cells are affected by EM waves.

In another exemplary aspect of the present disclosure, an integrated system for electromagnetic stimulating and electrical analyzing of cancer cells is disclosed. The integrated system for electromagnetic stimulating and electrical analyzing of cancer cells may be utilized as the integrated stimulating-analyzing set-up in an exemplary method 100 of the present disclosure for stimulating and analyzing of cancer cells.

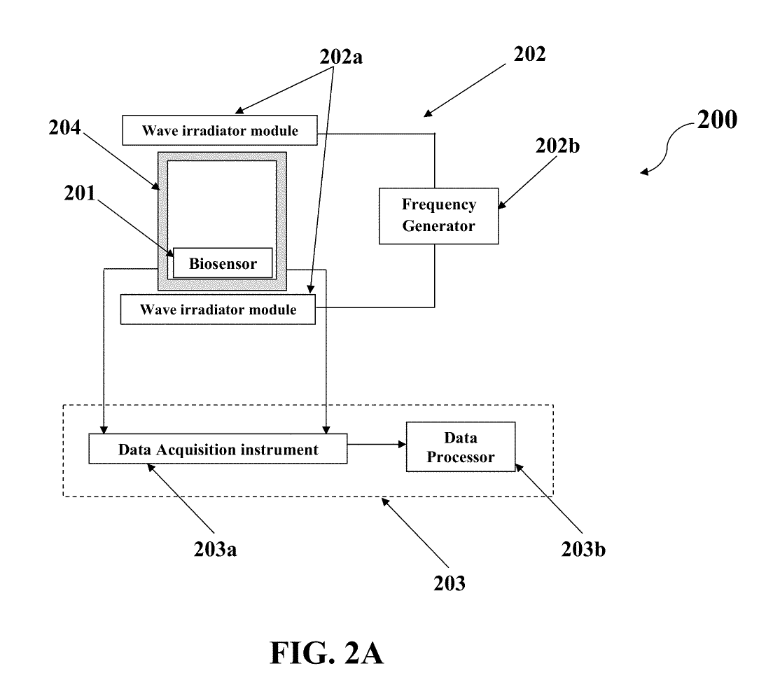

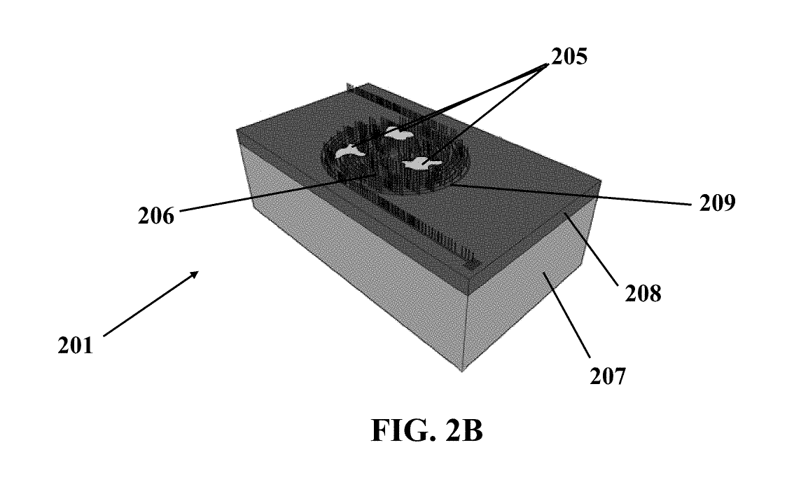

FIG. 2A shows an implementation of an exemplary integrated system 200 for electromagnetic stimulating and electrical analyzing of cancer cells. The system 200 may include: a biosensor 201 that may include an array of carbon nanotubes (CNTs), an electromagnetic wave exposure mechanism 202, and an electrical mechanism 203.

In some implementations, the biosensor 201 is placed in a sealed package 204 that may be placed within at least one wave irradiator module 202a. The sealed package 204 may be a sealing vessel, for example, a plaxy-glass set.

In some exemplary implementations, the electromagnetic wave exposure mechanism 202 may include a wave irradiator module 202a and a frequency generator 202b. The frequency generator 202b may be connected to at least one wave irradiator module 202a and may send electromagnetic (EM) waves to a sealed package 204 that may contain the biosensor 201.

In some implementations, the electrical mechanism 203 may include a data acquisition instrument 203a and a data processor 203b. The data acquisition instrument 203a may be configured to send an electrical signal to the biosensor 201 and receive an electrical response from the biosensor 201. The data processor 203b may be configured to process the received electrical signals from the biosensor 201. The biosensor 201, the data acquisition instrument 203a and the data processor 203b may be electrically connected.

FIG. 2B shows an example of a biosensor 201, in which a plurality of cancer cells 205 may be attached onto an array of carbon nanotubes (CNTs) 206 so that the CNTs 206 may be penetrated into the cancer cells 205, consistent with one or more exemplary embodiments of the present disclosure. The biosensor 201 may include an array of carbon nanotubes (CNTs) 206, for example, an array of VAMWCNTs. The CNTs 206 may be grown on a patterned region on the surface of biosensor 201, for example, a circular patterned region. Referring to FIG. 2B, the biosensor 201 may include a substrate layer 207, an insulator layer 208, a catalyst layer 209, and an array of carbon nanotubes (CNTs) 206.

In some implementations, the substrate layer 207 may include a layer of silicon (Si). Furthermore, the insulator layer 208 may include a layer of silicon dioxide (SiO₂) that may be formed on the substrate layer. In addition, the catalyst layer 209 may include a patterned layer of Nickel (Ni), for example, a circular patterned layer 209.

In some implementations, the array of carbon nanotubes (CNTs) 206 may be grown on the patterned catalyst layer 209. The CNTs 206 may include an array of vertically aligned multiwall carbon nanotubes (VAMWCNTs) grown on the patterned catalyst layer 209.

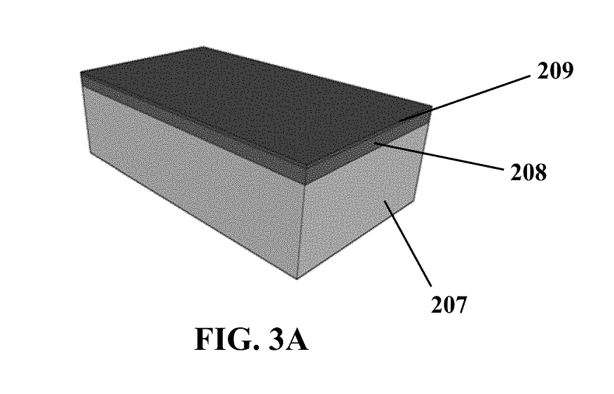

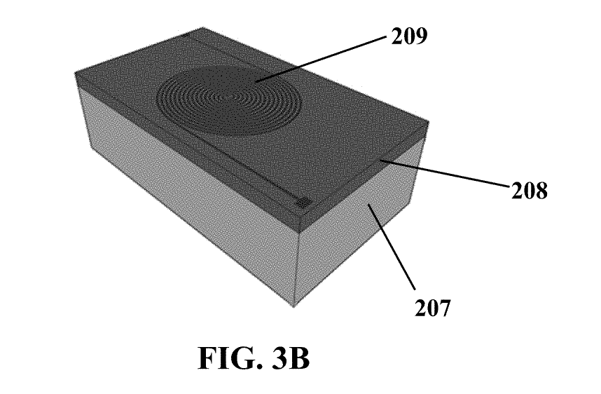

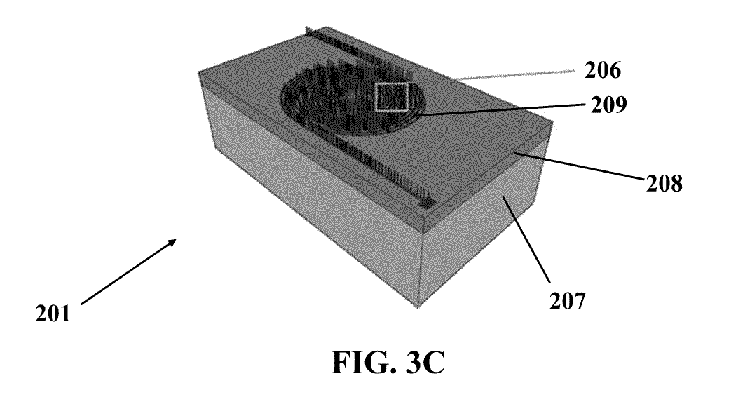

In an aspect, the biosensor 201 may be fabricated by a process method that is schematically shown in FIGS. 3A-3C. According to these figures, a substrate layer 207, for example, a Si chip may be supplied. Then, a dioxide insulator layer 208, for example, a SiO₂ layer may be formed and covered on the substrate layer 207. Subsequently, a catalyst layer 209, for example, a layer of Ni may be formed on the insulator layer 208 (FIG. 3A). As shown in FIG. 3B, the catalyst layer 209 may be patterned, for example, in a circular pattern. The array of CNTs, for example, an array of vertically aligned multiwall carbon nanotubes (VAMWCNTs) may be grown on the patterned catalyst layer 209 to form the biosensor 201 (FIG. 3C).

### EXAMPLES

### Example 1

### Biosensor Fabrication

In this example, at first, a Si wafer was cleaned by the standard Radio Corporation of America (RCA) method (RCA#1 method with NH₄OH:H₂O₂:H₂O solution and volume ratio of about 1:1:5), then rinsed in deionized (DI) water and after that, blow-dried by air. Thermal oxide was then grown on the wafers in wet oxide furnace at about 1050° C. with the assistance of H₂O (g) for about 2.5 hours. Then, a Ni thin film with the thickness of about 9 nm was deposited on the Si substrate using e-beam evaporation system at the temperature of about 120° C. and with depositing rate of about 0.1 Å/s. After that, the wafer was cut into pieces desired for the sensors (25 mm²). Then, the Ni layer was patterned in the shape of circular electrodes using standard photolithography process in which a thin layer (about 400 nm) of positive photoresist was spin coated on the surface. After illumination with Mask-Aligner System, the samples were chemically developed. Then, the Ni layer in undesired region was etched using Ni-etch solution (HNO₃:H₃PO₄:CH₃COOH, in amounts with a ratio about 3:3:1) and after that the photoresist was washed using acetone.

Finally, the samples were held in a direct-current plasma enhanced chemical vapor deposition reactor (DC-PECVD) to grow vertically aligned multi-walled carbon nano-tubes (VAMWCNT) on desired places. Using DC-PECVD, the samples were annealed at about 650° C. in a dynamic H₂ environment with a flow rate of about 35 standard cubic centimeters per minute (SCCM) for about 10 min to about 15 min. Thermally treated Ni layer was hydrogenated by plasma with a power density of about 5.5 W cm⁻² for about 5 min to obtain Ni nano-grains. The CNTs were grown on the Ni seeds in the same chamber containing a mixture of H₂ and C₂H₂ gases with flow rates of about 35 SCCM and about 5 SCCM at a temperature of about 650° C. and a pressure of about 0.28 kPa.

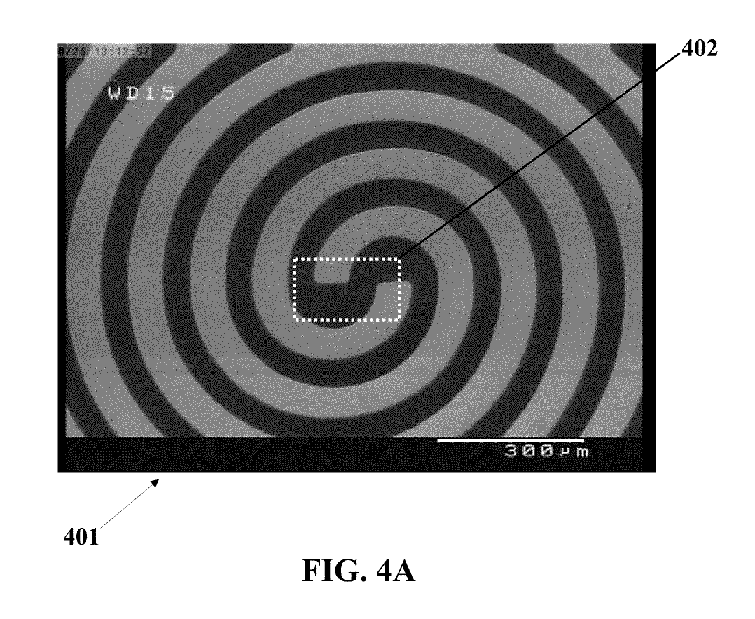

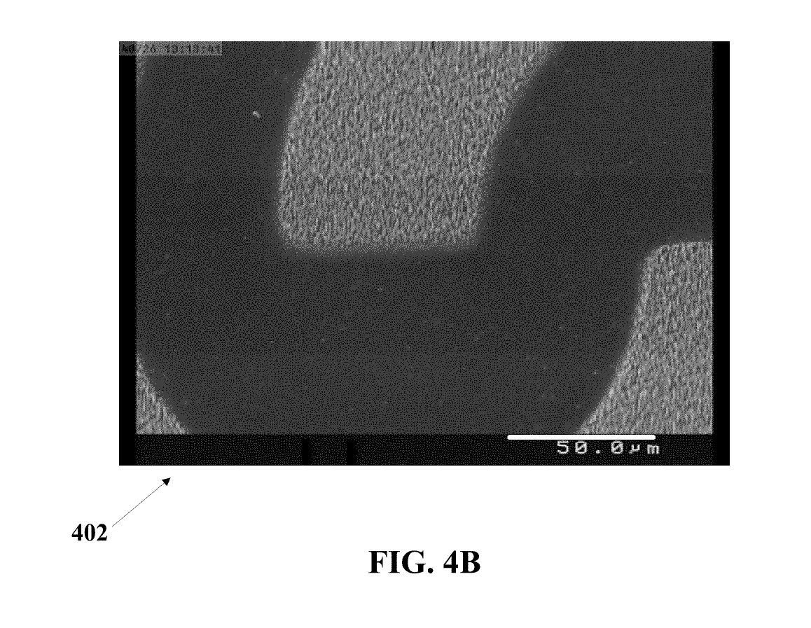

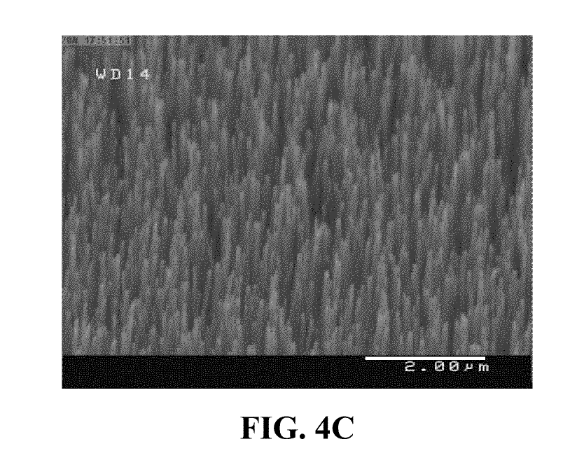

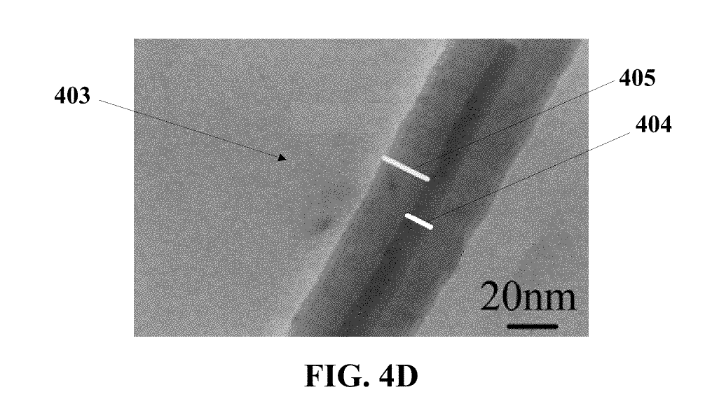

FIG. 4A shows a field emission scanning electron microscope (FESEM) micrograph of an example section 401 of the surface of a biosensor 201 including an array of grown CNTs on the patterned Ni layer. The size and geometry of nanotubes are presented in FIG. 4B-4D.

FIG. 4B shows a field emission scanning electron microscope (FESEM) micrograph of an example magnified section 402 in FIG. 4A, which illustrates an example array of the grown VAMWCNTs on the patterned Ni layer. FIG. 4C shows a field emission scanning electron microscope (FESEM) micrograph of an example grown array of CNTs. The width of the CNT covered micro-electrodes is about 70 μm with about 50 μm distance between. Highly ordered CNTs have been achieved with desired patterns and geometries. The average length of the CNTs is about 2.5 μm and the diameter of the CNTs is about 55 nm.

In FIG. 4D, a transmission electron microscope (TEM) image of an example of one CNT 403 is shown. As shown in this figure, the diameter of the CNT 403 is about 9 nm for diameter 404 to about 20 nm for diameter 405.

### Example 2

### Cell Culture on the Fabricated Biosensor

In this example, QUDB cell line was originally isolated from malignant human lung tissue. These cells were obtained from the standard cell Banks and they were maintained at about 37° C. (about 5% CO₂, about 95% clean air) in RPMI-1640 medium supplemented with about 5% fetal bovine serum, and about 1% penicillin/streptomycin. The fresh medium was replaced every other day. The cells were detached from the culture flask by trypsin and by use of a fresh media were cultured on the CNT covered electrodes of the fabricated biosensor, in connection with EXAMPLE 1. The cellular density was about 105 cells/ml and same for all the conditions and repetitions. During the tests, the samples were held in an incubator (for about 4 hours) without changing the media solution.

### Example 3

### CNT Penetration into Cells

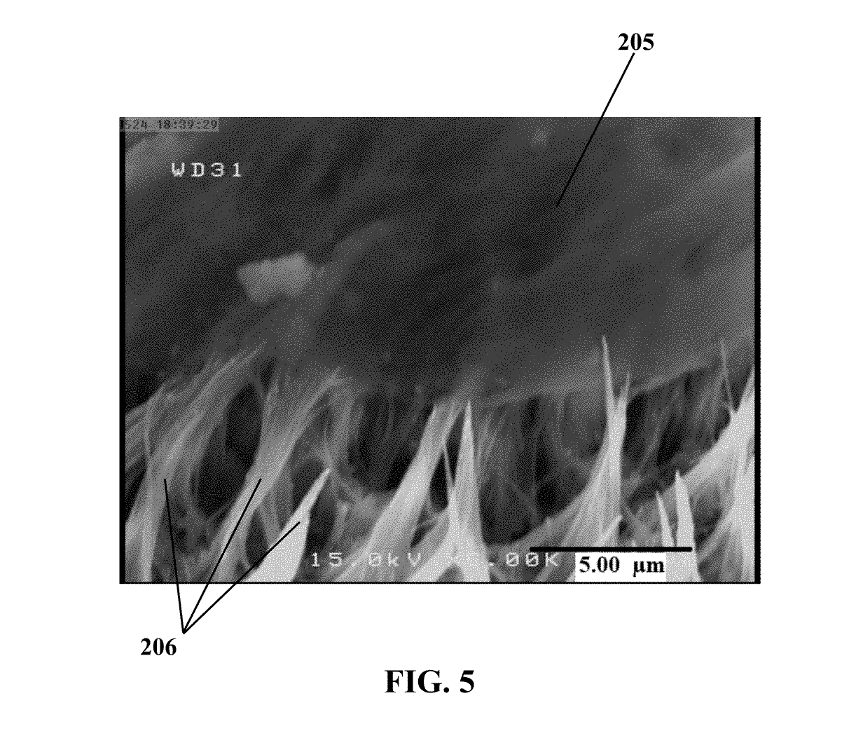

In order to be sure about CNTs penetration to the cells, some high resolution FESEM images are shown in FIG. 5. In this figure, presented the interaction interface between CNTs 206 and cellular membrane of an example QUDB cells 205. It is observable that the CNTs applied direct attachment and penetration to the membrane of QUDB cells 205. The tip of CNTs 206 directly penetrate into the membrane which could induce the electrical charges into the cells as a result of electromagnetic stimulation.

### Example 4

### Electromagnetic Stimulation/Electrical Monitoring of Cancer Cells

In this example, the cultured cancer cell lines on the CNTs array of the fabricated biosensor, according to EXAMPLEs 1-3, concurrently underwent electromagnetic stimulation and electrical monitoring/analysis. Prior to any contact to cell solution or EM wave stimulation, the fabricated sensors were electrically analyzed using NI DAQ (National Instrument Data Acquisition USB6323) impedance measurements. Then, the sensors were placed in plaxy-glass sets and QUDB cells suspended in media solution were inserted to the sets and cultured on the sensor surface. After about 2 hours, all the samples underwent impedance measurements. Immediately after that, half of them were irradiated by EM wave for about 5 min with different intensities in different experiments and the other half were held as controls. About 2 hours after that (about 4 hours away from the beginning of the process), all the samples underwent impedance measurements again (regarding that more than about 1.5 hours were required to observe the effect of EM stimulion cellular proliferation and mitosis). For 4 experimental condition, 4 individual set of biosensors were used. Each measurement was repeated 10 times. Statistical calculations were derived in each point of frequency.

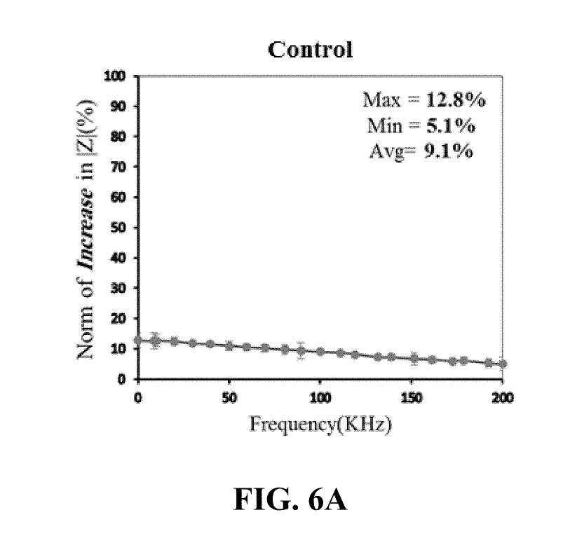

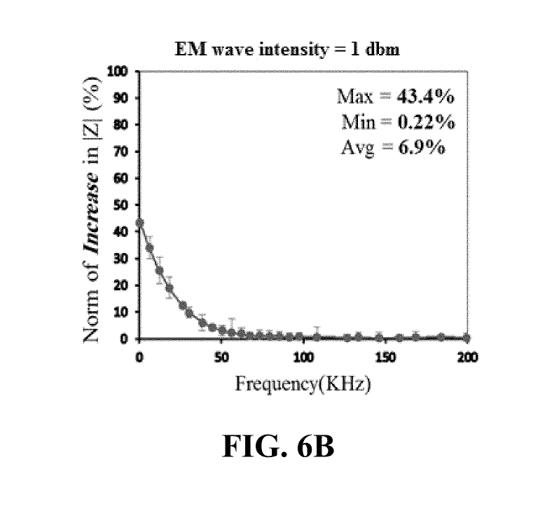

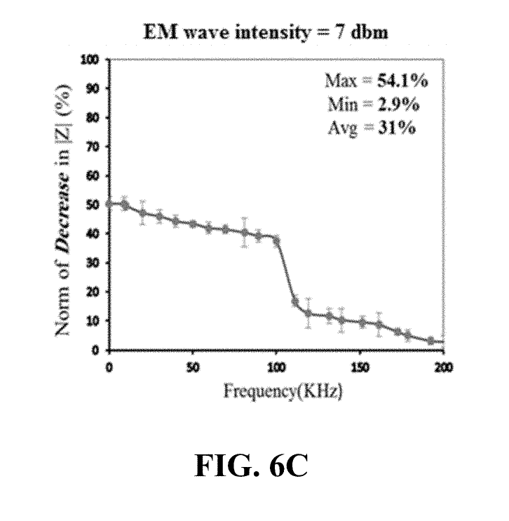

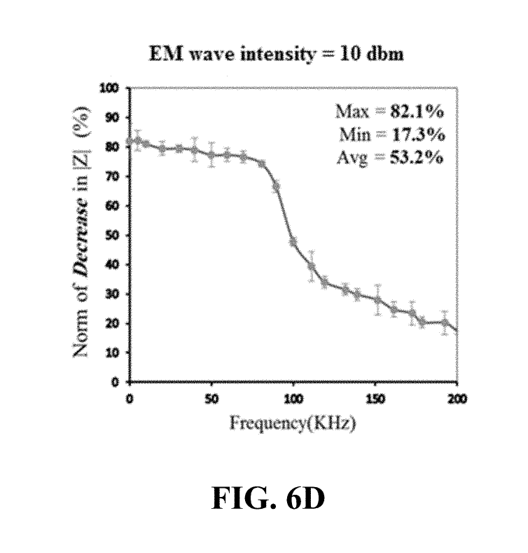

FIG. 6A shows an example curve of the norm of changes in impedance of QUDB cells (with the concentration of about 105 cells/ml) at about 2 hours and 4 hours after culturing on CNT covered circular shaped transducers for the control sample which was not irradiated. These results were compared to the similar example curves of the norm of impedance changes for samples that were exposed by about 940 MHz EM wave with the intensities of 1 dbm (FIG. 6B), 7 dbm (FIG. 6C) and 10 dbm (FIG. 6D). The rise or fall in the norm of impedance amplitude has been indicated in each figure separately. The impedance responses were normalized by the values achieved at the start time of measurement (2nd hour). In this regard, the normalized difference used in impedance figures would be equal to Eq. (1):

Z(4thhour)−Z(2ndhour)/Z(2ndhour)×100 Eq. (1)

Referring to FIGS. 6A, 6C and 6D, Impedance rise ranged from about 12.8% to about 5.1% in the frequencies between about 0.1 and about 200 kHz was observed for control sample (FIG. 6A). Meanwhile, impedance fall ranged from about 82.1% to about 17.3% for the sample that was irradiated by about 10 dbm EM wave (FIG. 6D) and about 54.1% to about 2.9% for the sample that was irradiated by about 7 dbm EM wave (FIG. 6C) was measured in the same range of frequencies after the same period of time. Considerable reduction in the power of EM wave (10 dbm in FIG. 6D vs. 7 dbm in FIG. 6C) showed a constructive effect on the impedance of the cells cultured on CNT electrodes (53.2% in FIG. 6D vs. 31% in FIG. 6C reduction in the mean impedance).

Referring to FIG. 6B, the impedance of the samples irradiated by about 1 dbm EM wave with similar parameters shows a rising trend. The measured increment was between about 43.4% and about 0.22% in the same range of frequencies comparable to the control sample (FIG. 6A).

It should be mentioned that increasing in the impedance of seeded cells confirms their appropriate adhesion and proliferation on the biosensor. Also, the increased impedance presents the ability of membrane in current flow blocking (beta dispersion phenomena) which are all directly correlated with higher vitality of the cells. Here, the impedance of destructed cells reduced (FIGS. 6C and 6D) in comparison with living ones (FIGS. 6A and 6B) because of the cells' degraded membrane. For a more precise analysis, with carefully following the norm of impedance variation diagrams (FIGS. 6A-D), it is obvious that the rate of impedance increment in the control sample (FIG. 6A), linearly decreased by increasing the scanning frequency. Meanwhile in the 10 dbm ablated sample (FIG. 6D), it is observed that the impedance decrease with a steady rate (about 80%) in lower frequencies up to about 90 kHz. Also, a semi-steady rate of decrease in the impedance for 7 dbm ablated sample (about 50-40%) (FIG. 6C) was measured in the same range of frequencies. This might be related to the fact that in the frequencies lower than about 100 kHz, the role of membrane properties is dominant in cell's electrical signal extraction. In 10 dbm ablated sample (FIG. 6D), the dielectric properties of the cells' membrane were noticeably degraded. So the membranes lost their ability in current blocking. Hence, a sharp steady decrease in the impedance of the cells up to about 100 kHz was observed. The similar evidences with minor strength might occur for 7 dbm ablated samples (FIG. 6C). However, when the cells were ablated to about 1 dbm EM wave (FIG. 6B), they continued their attachment and proliferation on CNTs leading to an increase in impedance of the biosensor. So the low intensity wave didn't induce noticeable perturbation to the cells. But, as an indication for destructive effects of the CNT assisted EM stimulation, the rate of impedance increment followed a sharp and non-linear reductive regime comparing to that of the control sample (FIG. 6A). It is known that the membrane impedance of a living cancerous cell (as a dielectric layer) decreases nonlinearly by increasing the frequency. However, impedance increment of control samples versus frequency followed a linear reductive regime. This might be attributed to the fact that the frequency dependent role of membrane capacitance in the overall impedance of our sensor was non-linear.

Furthermore, the norm in impedance phase changes for the same samples were analyzed. Phase diagram reflects the capacitance and dielectric properties of cultured cells. So it contains valuable data about the effect of stimulation on dielectric properties of the membrane. Phase progression in negative regime is the result of the cells' stronger dielectric properties.

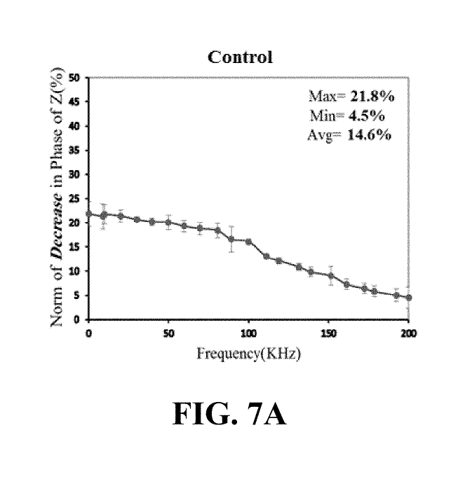

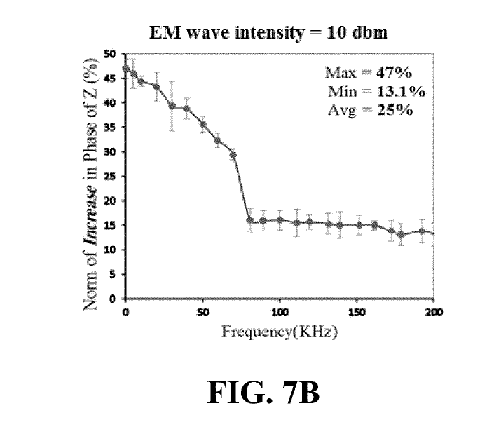

FIG. 7B shows the norm of changing in phase diagram of QUDB cells exposed by 10 dbm EM wave in comparison with the same diagram in FIG. 7A for non-irradiated control cells. There is a phase reduction ranged from about 21.8% to about 4.5% in the frequencies between about 0.1 and about 200 kHz for the control sample (FIG. 7A). In contrast, a phase increase ranged between about 47% and about 13.1% in the same range of frequencies is observed for the exposed sample (FIG. 7B). The phase values of a living cancer cell are negative and better attachment of the cells enhances the phase of the impedance sensor in negative regimes. In contrast, disruption in dielectric properties of the cells reduces the phase in negative regimes (lower in absolute magnitude). The comparative data shown in FIGS. 7A and 7B revealed the phase progression in negative regime for control samples as an indication of membrane dielectric strength and the cells' adhesion on the electrodes. In contrast, phase progression in positive regime for wave ablated cells indicated the functional disruption of membrane.

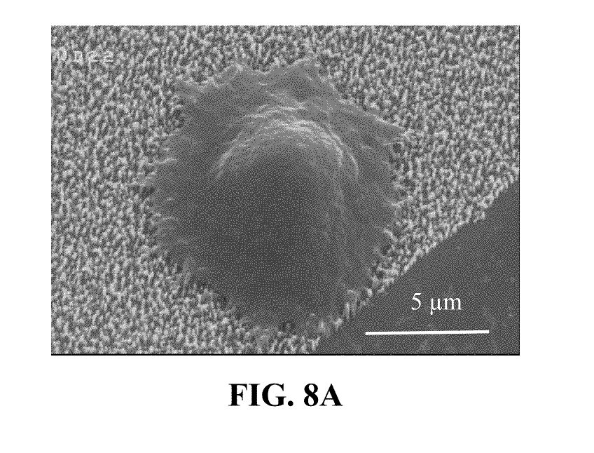

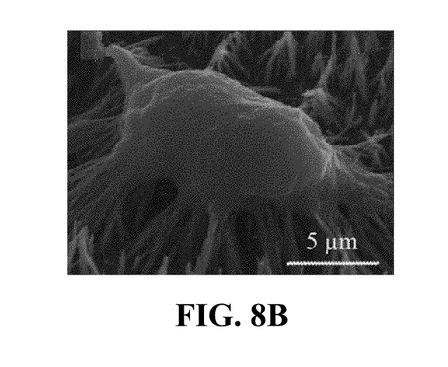

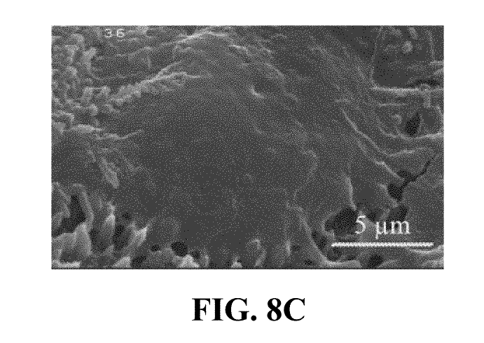

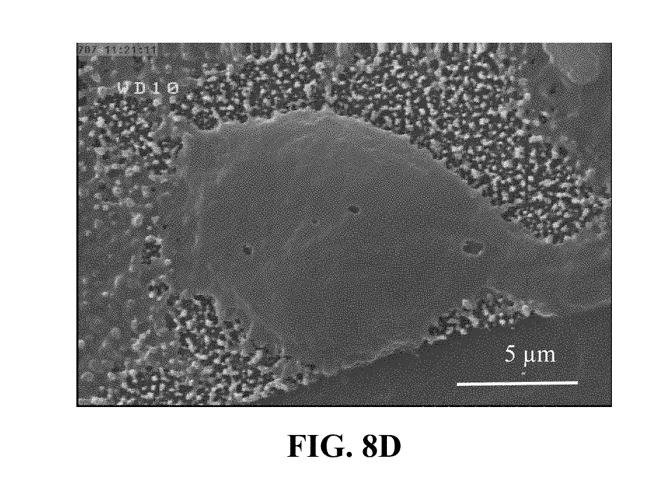

FIGS. 8A-D show field emission scanning electron microscope (FESEM) images of exemplary QUDB cells attached to the CNTs array for exemplary non-irradiated QUDB cells (control) (FIG. 8A) and for the samples ablated to a 1 dbm (FIG. 8B), 7 dbm (FIG. 8C) and 10 dbm (FIG. 8D) EM wave irradiation. It is understood that carbon nanotubes are local field enhancers and localized currents are created among irradiated CNTs. More importantly, electrical charges would be accumulated on the tips of such structures in response to external high frequency EM fields in ranges more than few MHz. As presented in FIG.s 8A-D, the tips of the CNTs were penetrated into the cell membrane. So the accumulated charges on top of the tips, initiated from the EM field, might induce some electrochemical variations into the cell, which could perturb the bioelectrical equilibrium of ions in cytoplasm and membrane regions. This might occur by ion exchange reactions between charges accumulated on tips of penetrated CNTs and aqueous ions in cells' inner parts that would cause perturbation in cellular functionality. Furthermore, induced currents among the CNTs by these charges, altered the transmembrane voltage and hence caused variations in membrane functions. Hence, CNTs induced considerable electrochemical non-equilibrium into the cells while tens of nanotubes are involved in stimulation of a single cancer cell as shown in FIGS. 8A-D.

It should be understood that reducing the intensity of EM wave results in considerable reduction in the cells' destruction. When the intensity of the wave is reduced to 1 dbm (about 50% lower power than 7 dbm), the destructive effects of such inductions were noticeably decreased and a 6.9% increase in the impedance of 1 dbm ablated cells seeded on CNT arrays about 2 hours after the wave ablation (FIGS. 6B and 6C), corroborates that the cells continued their proliferation. But such proliferation was lower than that of control samples (9.1% increment in mean impedance) (FIG. 6A) which reflected the minor destructive effect of low intensity wave ablation on the vitality of the cells penetrated by CNTs. It has been achieved that increased number of attached vital cells on the biosensor resulted in further current blockage and increased impedance responses. Additionally, better vitality of cancer cells leads to enhancement of their proliferation rate.

Referring again to FIGS. 8A-D, the presented images corroborated the destructive effects of high power EM wave on the cells seeded on CNT sensor. The shape, granularity and uniformity of the cells exposed by 7 dbm EM wave (FIG. 8C) and 10 dbm EM wave (FIG. 8D) were degraded with respect to samples exposed by 1 dbm EM wave (FIG. 8B). As observable in FIG. 8A, control cells homogenously spread on nanotubes, exhibited conformal adhesion.

### Example 5

### Vitality Assessment by MTT Assay

In this example, the cells vitality of EXAMPLE 4, was assessed by a MTT assay. Furthermore, the same MTT assay was carried out for two similar samples, in which the surface of biosensor was not covered by an array of CNTs, so that the cells were cultured on the Ni layer. One of these samples was used as a Ni control sample that was not irradiated and the second one was irradiated by about 10 dbm EM wave in the same conditions for the corresponding sample of CNTs-cultured cells. All samples were compared with a main control sample seeded on coverslip.

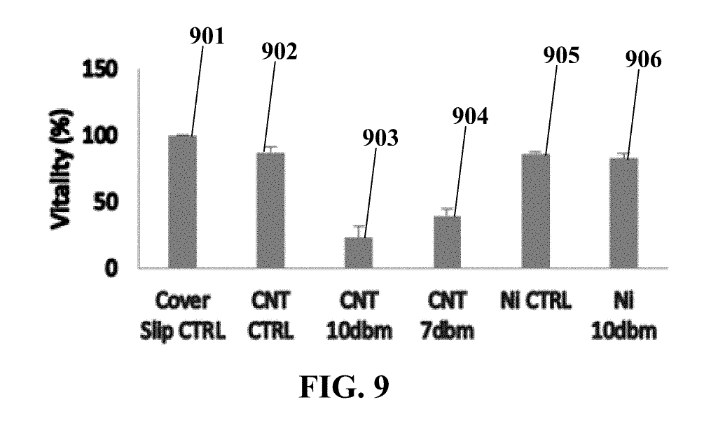

FIG. 9 shows the MTT results of the main control sample seeded on coverslip (901), cells cultured on CNT arrays (nonirradiated (902), irradiated by about 7 dbm waves (904) and irradiated by about 10 dbm waves (903)), cells cultured on Ni (nonirradiated (905) and irradiated by about 10 dbm waves (906)). The cells cultured on CNT arrays showed more than about 87% vitality for the non-irradiated cultured cells 902. Meanwhile the vitality of CNT mediated wave exposed cells were less than about 23% (903) about 2 hours after irradiation (for 5 min) by about 10 dbm waves; and about 40% (904) about 7 dbm waves. So the results coincided the impedance spectroscopy and SEM analysis. The MTT results of control (905) and wave irradiated cells (906) that were covered on Ni layer indicated more than about 85% vitality for control cells (905) and about 82% vitality for the wave irradiated cells (906). As such destruction was so minor (about 3% reduction in viability of wave irradiated cells) for Ni covered sample (without CNTs), the key role of CNTs in transferring and enhancing the EM waves and the resulted charge accumulations to the cells may be verified. Additionally, as MTT contains valuable data about the actual cell counts on the samples, the previous discussion in EXAMPLE 4 on proliferation and vitality of the control and ablated cells could be relied based on MTT assay.

What is claimed is:

1- A method for stimulating and analyzing of cancer cells, comprising:
preparing an integrated stimulating-analyzing set-up, including an array of carbon nanotubes (CNTs),
wherein a plurality of cancer cells attached onto the array of carbon nanotubes (CNTs),
measuring a first electrical response from the attached cancer cells;
applying an electromagnetic field on the attached cancer cells to stimulate cancer cells;
measuring a second electrical response from the stimulated cancer cells; and
detecting the vitality of the stimulated cancer cells by comparing the first and the second measured electrical responses.

2- The method according to claim 1, wherein detecting the vitality of the stimulated cancer cells comprises detecting that the vitality of the stimulated cancer cells is decreased if the second electrical response has a reversed trend versus the trend of the first electrical response in a same frequencies and intensities range of the applied electromagnetic field.

3- The method according to claim 1, wherein detecting the vitality of the stimulated cancer cells comprises detecting that the vitality of the stimulated cancer cells is decreased if the second electrical response has greater amounts versus the amounts of the first electrical response in a same range of applied frequencies and intensities.

4- The method according to claim 3, wherein the greater amounts of the second electrical response are at least 10 percent (10%) greater than the amounts of the first electrical response.

5- The method according to claim 1, wherein the carbon nanotubes (CNTs) include vertically aligned multiwall carbon nanotubes (VAMWCNTs).

6- The method according to claim 1, wherein the integrated stimulating-analyzing set-up includes:
a biosensor, including the array of CNTs grown on a chip,
an electrical analyzing device, including:
a data acquisition instrument, configured to send an electrical signal to the biosensor and receive an electrical response from the biosensor; and
a data processor, configured to process the received electrical signals, wherein the biosensor, the data acquisition instrument and the data processor are electrically connected; and
an electromagnetic wave exposure device, including:
a wave irradiator module; and
a frequency generator,
wherein the frequency generator is connected to the wave irradiator module.

7- The method according to claim 1, wherein the preparing the integrated stimulating-analyzing set-up include:
holding a biosensor in a sealed package,
wherein the biosensor includes the array of CNTs;
connecting the biosensor to an electrical analyzing device;
inserting a solution of cancer cells into the sealed package and on the array of CNTs;
placing the sealed package in an electromagnetic wave exposure device; and
placing the electromagnetic wave exposure device including the sealed package in an incubator.

8- The method according to claim 7, wherein the solution of cancer cells includes a plurality of cancer cells suspended in a cell-culture media.

9- The method according to claim 1, wherein the cancer cells include lung cancer cells. 10- The method according to claim 1, wherein the cancer cells include lung cancer cell

lines.

11- The method according to claim 1, wherein the first and the second electrical responses are measured within a determined frequency range between 0.1 and 500 kHz.

12- The method according to claim 11, wherein the first and the second measured electrical responses include a first set and a second set of electrical impedance values measured in the determined frequency range.

13- The method according to claim 11, wherein the first and the second measured electrical responses include a first set and a second set of electrical impedance values measured in the determined frequency range.

14- The method according to claim 1, wherein the electromagnetic field is applied with an intensity in a range of 1 dbm to 20 dbm.

15- The method according to claim 1, wherein the electromagnetic field is applied with a frequency of 940 MHz.

16- An integrated system for electromagnetic stimulating and electrical analyzing of cancer cells, comprising:
a biosensor, including an array of carbon nanotubes (CNTs);
an electromagnetic wave exposure mechanism, including:
a wave irradiator module; and
a frequency generator,
wherein the frequency generator is connected to the wave irradiator module and the biosensor is placed in a sealed package that is placed within the wave irradiator module; and
an electrical mechanism, including:
a data acquisition instrument, configured to send an electrical signal to the biosensor and receive an electrical response from the biosensor; and
a data processor, configured to process the received electrical signals, wherein the biosensor, the data acquisition instrument and the data processor are electrically connected and configured to detect the vitality of the stimulated cancer cells by comparing the first and the second measured electrical responses.

17- The system according to claim 16, wherein the biosensor transfers electromagnetic stimulation to the cancer cells and acquire electrical signal from cancer cells, concurrently.

18- The system according to claim 16, wherein the carbon nanotubes (CNTs) include vertically aligned multiwall carbon nanotubes (VAMWCNTs).

19- The method according to claim 16, wherein the sealed package includes a plaxy-glass set.

20- The system according to claim 16, wherein the biosensor comprises:
a substrate layer, wherein the substrate layer includes a layer of silicon (Si);
an insulator layer, wherein the insulator layer includes a layer of silicon dioxide (SiO₂) formed on the substrate layer;
a catalyst layer, wherein the catalyst layer includes a patterned layer of Nickel (Ni); and
an array of carbon nanotubes (CNTs) on the patterned layer, including an array of vertically aligned multiwall carbon nanotubes (VAMWCNTs) grown on the patterned catalyst layer.
